SEQUENCE LISTING (6 sequences)

SEQ ID NO: 1 - DNA, 1101 bp, Homo sapiens
  source 1..1101: mol_type = genomic DNA
ttaaccccaggaaaccccaagtgggaacgcactaacttgacctacaggattcgaaactataccccacagctgtcagaggctgaggtagaaagagctatcaaggatgcctttgaactctggagtgttgcatcacctctcatcttcaccaggatctcacagggagaggcagatatcaacattgctttttaccaaagagatcacggtgacaattctccatttgatggacccaatggaatccttgctcatgcctttcagccaggccaaggtattggaggagatgctcattttgatgccgaagaaacatggaccaacacctccgcaaattacaacttgtttcttgttgctgctcatgaatttggccattctttggggctcgctcactcctctgaccctggtgccttgatgtatcccaactatgctttcagggaaaccagcaactactcactccctcaagatgacatcgatggcattcaggccatctatggactttcaagcaaccctatccaacctactggaccaagcacacccaaaccctgtgaccccagtttgacatttgatgctatcaccacactccgtggagaaatacttttctttaaagacaggtacttctggagaaggcatcctcagctacaaagagtcgaaatgaattttatttctctattctggccatcccttccaactggtatacaggctgcttatgaagattttgacagagacctcattttcctatttaaaggcaaccaatactgggctctgagtggctatgatattctgcaaggttatcccaaggatatatcaaactatggcttccccagcagcgtccaagcaattgacgcagctgttttctacagaagtaaaacatacttctttgtaaatgaccaattctggagatatgataaccaaagacaattcatggagccaggttatcccaaaagcatatcaggtgcctttccaggaatagagagtaaagttgatgcagttttccagcaagaacatttcttccatgtcttcagtggaccaagatattacgcatttgatcttattgctcagagagttaccagagttgcaagaggcaataaatggcttaactgtagatatggc

SEQ ID NO: 2 - protein, 367 aa, Homo sapiens
LTPGNPKWERTNLTYRIRNYTPQLSEAEVERAIKDAFELWSVASPLIFTRISQGEADINIAFYQRDHGDNSPFDGPNGILAHAFQPGQGIGGDAHFDAEETWTNTSANYNLFLVAAHEFGHSLGLAHSSDPGALMYPNYAFRETSNYSLPQDDIDGIQAIYGLSSNPIQPTGPSTPKPCDPSLTFDAITTLRGEILFFKDRYFWRRHPQLQRVEMNFISLFWPSLPTGIQAAYEDFDRDLIFLFKGNQYWALSGYDILQGYPKDISNYGFPSSVQAIDAAVFYRSKTYFFVNDQFWRYDNQRQFMEPGYPKSISGAFPGIESKVDAVFQQEHFFHVFSGPRYYAFDLIAQRVTRVARGNKWLNCRYG

SEQ ID NO: 3 - DNA, 1194 bp, synthetic construct
  source 1..1194: mol_type = other DNA
atgttctccctgaagacgcttccatttctgctcttactccatgtgcagatttccaaggccttaaccccaggaaaccccaagtgggaacgcactaacttgacctacaggattcgaaactataccccacagctgtcagaggctgaggtagaaagagctatcaaggatgcctttgaactctggagtgttgcatcacctctcatcttcaccaggatctcacagggagaggcagatatcaacattgctttttaccaaagagatcacggtgacaattctccatttgatggacccaatggaatccttgctcatgcctttcagccaggccaaggtattggaggagatgctcattttgatgccgaagaaacatggaccaacacctccgcaaattacaacttgtttcttgttgctgctcatgaatttggccattctttggggctcgctcactcctctgaccctggtgccttgatgtatcccaactatgctttcagggaaaccagcaactactcactccctcaagatgacatcgatggcattcaggccatctatggactttcaagcaaccctatccaacctactggaccaagcacacccaaaccctgtgaccccagtttgacatttgatgctatcaccacactccgtggagaaatacttttctttaaagacaggtacttctggagaaggcatcctcagctacaaagagtcgaaatgaattttatttctctattctggccatcccttccaactggtatacaggctgcttatgaagattttgacagagacctcattttcctatttaaaggcaaccaatactgggctctgagtggctatgatattctgcaaggttatcccaaggatatatcaaactatggcttccccagcagcgtccaagcaattgacgcagctgttttctacagaagtaaaacatacttctttgtaaatgaccaattctggagatatgataaccaaagacaattcatggagccaggttatcccaaaagcatatcaggtgcctttccaggaatagagagtaaagttgatgcagttttccagcaagaacatttcttccatgtcttcagtggaccaagatattacgcatttgatcttattgctcagagagttaccagagttgcaagaggcaataaatggcttaactgtagatatggcggatccaactggagccaccctcagttcgagaag

SEQ ID NO: 4 - protein, 398 aa, synthetic construct
MFSLKTLPFLLLLHVQISKALTPGNPKWERTNLTYRIRNYTPQLSEAEVERAIKDAFELWSVASPLIFTRISQGEADINIAFYQRDHGDNSPFDGPNGILAHAFQPGQGIGGDAHFDAEETWTNTSANYNLFLVAAHEFGHSLGLAHSSDPGALMYPNYAFRETSNYSLPQDDIDGIQAIYGLSSNPIQPTGPSTPKPCDPSLTFDAITTLRGEILFFKDRYFWRRHPQLQRVEMNFISLFWPSLPTGIQAAYEDFDRDLIFLFKGNQYWALSGYDILQGYPKDISNYGFPSSVQAIDAAVFYRSKTYFFVNDQFWRYDNQRQFMEPGYPKSISGAFPGIESKVDAVFQQEHFFHVFSGPRYYAFDLIAQRVTRVARGNKWLNCRYGGSNWSHPQFEK

SEQ ID NO: 5 - DNA, 1404 bp, Homo sapiens
  source 1..1404: mol_type = genomic DNA
atgttctccctgaagacgcttccatttctgctcttactccatgtgcagatttccaaggcctttcctgtatcttctaaagagaaaaatacaaaaactgttcaggactacctggaaaagttctaccaattaccaagcaaccagtatcagtctacaaggaagaatggcactaatgtgatcgttgaaaagcttaaagaaatgcagcgattttttgggttgaatgtgacggggaagccaaatgaggaaactctggacatgatgaaaaagcctcgctgtggagtgcctgacagtggtggttttatgttaaccccaggaaaccccaagtgggaacgcactaacttgacctacaggattcgaaactataccccacagctgtcagaggctgaggtagaaagagctatcaaggatgcctttgaactctggagtgttgcatcacctctcatcttcaccaggatctcacagggagaggcagatatcaacattgctttttaccaaagagatcacggtgacaattctccatttgatggacccaatggaatccttgctcatgcctttcagccaggccaaggtattggaggagatgctcattttgatgccgaagaaacatggaccaacacctccgcaaattacaacttgtttcttgttgctgctcatgaatttggccattctttggggctcgctcactcctctgaccctggtgccttgatgtatcccaactatgctttcagggaaaccagcaactactcactccctcaagatgacatcgatggcattcaggccatctatggactttcaagcaaccctatccaacctactggaccaagcacacccaaaccctgtgaccccagtttgacatttgatgctatcaccacactccgtggagaaatacttttctttaaagacaggtacttctggagaaggcatcctcagctacaaagagtcgaaatgaattttatttctctattctggccatcccttccaactggtatacaggctgcttatgaagattttgacagagacctcattttcctatttaaaggcaaccaatactgggctctgagtggctatgatattctgcaaggttatcccaaggatatatcaaactatggcttccccagcagcgtccaagcaattgacgcagctgttttctacagaagtaaaacatacttctttgtaaatgaccaattctggagatatgataaccaaagacaattcatggagccaggttatcccaaaagcatatcaggtgcctttccaggaatagagagtaaagttgatgcagttttccagcaagaacatttcttccatgtcttcagtggaccaagatattacgcatttgatcttattgctcagagagttaccagagttgcaagaggcaataaatggcttaactgtagatatggctga

SEQ ID NO: 6 - protein, 467 aa, Homo sapiens [sequence omitted]

(19) United States
(12) Patent Application Publication
(10) Pub. No.: US 2023/0277667 A1
(43) Pub. Date: Sep. 7, 2023

(54) COMPOSITIONS AND METHODS OF USE OF GENETICALLY MODIFIED IMMUNE CELLS EXPRESSING MATRIX METALLOPEPTIDASE

(71) Applicant: University of Houston System, Houston, TX (US)

(72) Inventors: Navin Varadarajan, Houston, TX (US); Irfan Naseem Bandey, Houston, TX (US)

(21) Appl. No.: 17/984,169

(22) Filed: Nov. 9, 2022

Related U.S. Application Data
(60) Provisional application No. 63/263,818, filed on Nov. 9, 2021.

(51) Int. Cl. A61K 39/00; C12N 9/50; A61K 39/395; A61K 31/65; A61K 31/704; A61K 31/5377; A61K 31/436; A61K 31/517; A61K 31/4545; A61P 35/00

(52) U.S. Cl. CPC A61K 39/4631; A61K 31/436; A61K 31/4545; A61K 31/517; A61K 31/5377; A61K 31/65; A61K 31/704; A61K 39/3955; A61K 39/4611; A61K 39/4637; A61P 35/00; C12N 9/50; C12Y 304/24034

(57) ABSTRACT

Provided herein are compositions containing genetically modified immune cells that express a ECM degrading enzyme, such as a matrix metallopeptidase. Overexpression of mature matrix metallopeptidase improves the tumor infiltrating capabilities of immune cells against solid tumors, including T-cells, tumor infiltrating lymphocytes, CAR macrophages, and NK cells (with and without a chimeric antigen receptor). Methods include administering CAR T or NK cells modified to express mature matrix metallopeptidase 8 to a subject with a solid tumor to improve the efficacy of the CAR T/NK cell therapy.

### SEQUENCE LISTING

[000.1] The instant application contains a Sequence Listing which has been submitted electronically in XML format and is hereby incorporated by reference in its entirety. Said XML copy, created on Dec. 12, 2022, is named U108275_1010US1_3_6_SL.xml and is 11,182 bytes in size. The Sequence Listing does not extend beyond the scope of the specification, and does not add new matter.

### TECHNICAL FIELD

The disclosure relates to compositions and methods of use of genetically modified immune cells expressing a matrix metallopeptidase (MMP). More specifically, these compositions and methods include genetically modified immune cells expressing a mature matrix metallopeptidase 8 (mMMP8) that are administered as a therapeutic regimen against solid tumors.

### BACKGROUND

In the last decade, chimeric antigen receptor (CAR) T-cell therapy has shown remarkable success in hematological cancers. However, the clinical experience with solid tumors has not been equally encouraging. In solid tumors, the presence of extracellular matrix (ECM) is one of the key components that limits the efficacy of CAR T cell therapy. ECM provide stiff architectural support to solid tumors acting as a physical barrier, preventing the entry and killing of tumor specific immune cells inside the tumor mass. Moreover, ECM has been reported to prevent T cells infiltration in breast, lung and pancreas carcinoma. Excess production of various constituents of the ECM such as hyaluronan and collagen is correlated with poor prognosis in several tumors.

### SUMMARY

Applicant has recognized a need to modify and improve currently available immune cell therapy for patients with solid tumors. To eliminate tumors, adoptively transferred lymphocytes have been modified to home to the tumors before they can execute their effector functions. For example, the CAR T-cells and NK cells of this present disclosure have been genetically modified to overexpress the mature form of MMP8. Both in vitro and animal studies confirmed that MMP8 overexpressing CAR T/NK cells showed improved tumor cell killing efficacy compared to CAR T-cells alone. Expression of mMMP8 can be used to overcome the hurdles of current CAR T/NK cell therapy especially in patients with stroma-rich solid tumors by overcoming the physical barriers posed by ECM. Embodiments include a composition containing genetically modified immune cells expressing a mature form of a matrix metallopeptidase. The matrix metallopeptidase can be a matrix metallopeptidase 8. In certain embodiments, the genetically modified immune cells contain a mammalian cell expression construct with a nucleic acid encoding the matrix metallopeptidase. In certain embodiments, the nucleic acid encoding the matrix metallopeptidase contains SEQ ID NO. 1. Embodiments include a composition comprising genetically modified immune cells expressing a mature form of a matrix metallopeptidase corresponding to SEQ ID NO. 2. In certain embodiments, the genetically modified immune cells contain a mammalian cell expression construct with a nucleic acid corresponding to SEQ ID NO. 3, which includes a nucleotide sequence for a signal peptide, a nucleotide sequence of a mature MMP8, a BamHI restriction site sequence, and a nucleotide sequence streptavidin tag. Embodiments include a composition comprising genetically modified immune cells expressing a protein corresponding to SEQ ID NO. 4, which includes a signal peptide, a mature MMP8, a peptide linker, and a streptavidin tag.

Embodiments include the genetically modified immune cells containing a mRNA construct for expression of the matrix metallopeptidase. The genetically modified immune cells can be one or more of T cells, Natural Killer cells, Natural Killer T cells, or B cells. The genetically modified immune cells can include myeloid cells. The genetically modified immune cells can be further modified to express a chimeric antigen receptor. The immune cells can be isolated from peripheral blood or from human tumors. The genetically modified immune cells can be further modified to express a cytokine.

Embodiments include a pharmaceutical product containing a composition including genetically modified immune cells expressing a mature form of a matrix metallopeptidase for use in a method of a treatment of a solid tumor in a patient. The solid tumor can be associated with brain cancer, breast cancer, lung cancer, colorectal cancer, prostate cancer, or cervical cancer. The pharmaceutical product can be administered to a patient who has been administered a chemotherapeutic agent or who is being administered a chemotherapeutic agent concurrently. The chemotherapeutic agent can be doxycycline.

Embodiments include methods of treating a solid tumor in a patient by administering a composition containing genetically modified immune cells expressing a mature form of a matrix metallopeptidase. Embodiments include methods of treating a solid tumor in a patient by administering a composition containing genetically modified immune cells expressing a mature form of a matrix metallopeptidase and a chimeric antigen receptor. Embodiments include methods of treating a solid tumor in a patient by administering a composition containing genetically modified immune cells expressing a mature form of a matrix metallopeptidase and a cytokine. The method can further include administering a chemotherapeutic agent to the patient.

Embodiments include methods of treating a solid tumor in a patient by administering a composition containing chimeric antigen receptor-expressing T-cells modified to express a mature form of a matrix metallopeptidase 8. Embodiments include methods of treating a solid tumor in a patient by administering a composition containing chimeric antigen receptor-expressing natural killer cells modified to express a mature form of a matrix metallopeptidase 8. The solid tumor can be associated with brain cancer, breast cancer, lung cancer, colorectal cancer, prostate cancer, or cervical cancer. In certain embodiments, the method further includes the step of administering a chemotherapeutic agent. The chemotherapeutic agent can be one or more of doxycycline, doxorubicin, gefitinib, erlotinib, everolimus, afatinib, and crizotinib. In certain embodiments, the method further includes the step of administering an immunotherapeutic agent. The immunotherapeutic agent is one or more of an anti-PD1 (programmed cell death protein 1) antibody, anti-PDL1 (programmed death-ligand 1) antibody, anti-CTLA4 (Cytotoxic T-Lymphocyte Associated Protein 4) antibody, anti-LAG3 (Lymphocyte-activation gene 3) antibody, and an anti-TIM-3 (T cell immunoglobulin and mucin domain-3) antibody.

### BRIEF DESCRIPTION OF THE DRAWINGS

Embodiments will be readily understood by the following detailed description in conjunction with the accompanying drawings. To facilitate this description, like reference numerals designate like structural elements or procedures in a method. Embodiments are illustrated by way of example and not by way of limitation in the figures of the accompanying drawings.

FIG. 1A is a photographic representation of the data from a RT PCR assay of monocytes and T-cells upon activation at different time intervals. FIG. 1B is a graphical representation of the results from a Matrigel invasion assay for Monocytes and activated T-cells. FIG. 1C is a photographic representation of the data from a Western Blot analysis for secreted mMMP8 expression in conditioned media of activated T-cells.

FIG. 2A is a diagrammatic representation of the CAR construct containing the mMMP8. FIGS. 2B - 2E are representations of the phenotyping of CAR T-cells vs mMMP8-CAR T-cells. FIG. 2B and FIG. 2C are flow plots capturing the expression of the CD3 in CAR T-cells and mMMP8-CAR T-cells, respectively. FIG. 2D and FIG. 2E are flow plots capturing the expression of the CD8 in CAR T-cells and mMMP8-CAR T-cells, respectively. Fluorescent intensity are the units for all flow plots. FIG. 2F and FIG. 2G are representations of flow cytometry evaluation of CD62L and CD45 RA in CAR T-cells and mMMP8-CAR T-cells, respectively, and FIG. 2H and FIG. 2I are representations of flow cytometry evaluation of CD62L and CD45 RO in CAR T-cells and mMMP8-CAR T-cells, respectively. FIG. 2J and FIG. 2K are graphical representations of the results from the phenotyping of NALM6 and SKOV-3 cells for surface CD19 expression. FIG. 2L is a graphical representation of FF-Luc expression in NALM6 and SKOV-3 cells. FIG. 2M is a diagrammatic representation of the set-up for the killing assay upon co-culture of effector and targets in 96-well plate using D-luciferin substrate. FIG. 2N and FIG. 2O are graphical representations of the results from the killing assay upon co-culture of CAR T-cells and MMP8-CD19R-41BBz CAR T cells in the presence of NALM6 and SKOV-3 cells, respectively. FIG. 2P is a diagrammatic representation of the set-up for the killing assay in Matrigel performed in 96-well plate. Tumor cells were seeded in Matrigel and CAR T-cells were laid on top of the solidified Matrigel. FIG. 2Q and FIG. 2R are graphical representations of the results from the killing assay of CAR T-cells and MMP8-CD19R-41BBz CAR T cells laid on top of the solidified Matrigel containing NALM6 and SKOV-3 cells, respectively. FIG. 2S is a diagrammatic representation of the set-up for the killing assay using Boyden chamber. Tumor cells were seeded in the bottom chamber and CAR T-cells were plated in the Matrigel coated upper chamber. FIG. 2T and FIG. 2U are graphical representations of the results from the killing assay of CAR T-cells and MMP8-CD19R-41BBz CAR T cells in the in the Matrigel coated upper chamber above NALM6 and SKOV-3 cells, respectively, seeded in the bottom chamber.

FIGS. 3A - 3C are photographs representation of the in vivo imaging data demonstrating the enhancement of tumor infiltration and improvement of overall survival in xenograft tumor models, both in control mice (FIG. 3A) and in mice with CD19 CAR T-cells (FIG. 3B) and with CD19 CAR T-cells co-expressing mMMP8 (FIG. 3C) over a time period. FIG. 3D is a graphical representation of the tumor growth curve in control mice and in mice with CD19 CAR T-cells and with CD19 CAR T-cells co-expressing mMP8.

FIG. 4 is a graphical representation of the results from the killing assay using Boyden chamber. MMP8 expressing CAR NK-cells exhibited greater migratory capability across Matrigel compared to CAR NK-cells alone.

### DETAILED DESCRIPTION

Currently available CAR T-cell design for solid tumors has an antigen specific receptor consisting of a single-chain variable fragment (scFv) fused to transmembrane domain followed by a co-signaling domain and CD3z. This design has been efficiently used for treating hematological malignancies. However, its effectiveness in treating solid tumors has not been encouraging in part due to presence of ECM around solid tumors, which acts as a barrier preventing the migration of T-cells into tumor mass to kill the tumor cells. Any barrier that prevents T cell infiltration will render T-cell based therapies ineffective regardless of their functional capacity. Despite remarkable success in hematological cancers, the clinical experience with solid tumors has not been equally encouraging. For example, the ECM and collagen fibers produced by tumor cells and tumor associated fibroblasts has been shown to prevent T-cell infiltration in breast, lung and pancreatic cancers. Successful extravasation of T-cells into the tumor mass requires them to actively degrade the various components of ECM including heparan sulphate proteoglycans and collagen, without which they would be unable to access tumor cells and exert their antitumor effects.

Provided here are embodiments of genetically modified immune cells expressing an extracellular matrix degrading enzyme. Embodiments of ECM-degrading group of enzymes include matrix metalloproteinases (MMPs). The MMPs belong to a larger family of proteases known as the metzincin superfamily. They are metalloproteinases that are calcium-dependent zinc-containing endopeptidases. These MMPs are a group of enzymes that are capable of proteolysis of almost all ECM components and are characterized by their function as the collagenases, the gelatinases, the stromelysins, and the membrane-type MMPs. Additionally, these enzymes can cleave several cell surface receptors, chemokines, and cytokines. These functions enable MMPs to play a key role in the migration of normal and transformed cells inside the body, modulate cell proliferation, differentiation, angiogenesis, and apoptosis. MMPs have been proposed as biomarkers and therapeutic targets for various cancers. MMP 1 and 3 may serve as potential biomarkers in breast cancer development. Similarly, MMP1 and MMP9 may serve as potential prognostic biomarkers and targets for uveal melanoma. MMP11 has been suggested as a prognostic biomarker in pancreatic cancer.

In certain embodiments, the genetically modified immune cells contain a mammalian cell expression construct with a nucleic acid encoding the matrix metallopeptidase. In certain embodiments, the nucleic acid encoding the matrix metallopeptidase contains SEQ ID NO. 1. Embodiments include a composition comprising genetically modified immune cells expressing a mature form of a matrix metallopeptidase corresponding to SEQ ID NO. 2. In certain embodiments, the genetically modified immune cells contain a mammalian cell expression construct with a nucleic acid corresponding to SEQ ID NO. 3, which includes a nucleotide sequence for a signal peptide, a nucleotide sequence of a mature MMP8, a BamHI restriction site sequence, and a nucleotide sequence streptavidin tag. Embodiments include a composition comprising genetically modified immune cells expressing a protein corresponding to SEQ ID NO. 4, which includes a signal peptide, a mature MMP8, a peptide linker, and a streptavidin tag. The immune cells can be T-cells, tumor infiltrating lymphocytes, macrophages, or NK cells, each of the foregoing with and without being modified to express a chimeric antigen receptor. The genetically modified immune cells are administered to patients with a solid tumor. The solid tumor can be associated with brain cancer, breast cancer, lung cancer, colorectal cancer, prostate cancer, or cervical cancer. In particular embodiments, the genetically modified immune cells are administered to a patient who has received, is receiving, or will receive an additional cancer treatment, such as one that includes chemotherapy, immunotherapy, radiation, surgery, hormone therapy, or a combination thereof.

MMP8 cleaves triple helix type I collagen and various other ECM and non-ECM substrates. Studies using MMP8 knockout mice, mammary carcinoma showed accelerated tumor formation and tumor vascularity. Similarly, MMP8 overexpression has been shown to promote tumor cell adhesion to ECM. These findings support the antitumor role of MMP8 in stroma rich tumors. Embodiments include genetically modified immune cells expressing mature MMP8. MMP8 has a broad range of targets in the ECM including aggrecan, gelatins and, collagen. Studies show MMP8 has tumor protective role in melanoma and reduced the incidence of synchronous lymph node metastases in breast cancer. MMP8 is thus a validated candidate for enabling the targeted degradation of ECM.

Embodiments include genetically modified T or Natural Killer (NK) cells with MMPs. In certain embodiments, these genetically modified T or NK cells express MMP8. Neither T nor NK cells express appreciable amounts of MMP8 endogenously. In enforcing the recombinant overexpression of MMP8 in T/NK cells, Applicant recognized the need to modify the MMP8 to only express the mature form, which is the arrived at by cleavage of the propeptide. For example, the complete mRNA sequence is available at the National Library of Medicine’s GenBank database under the accession identifier: NM_002424.3, MMP8 is natively synthesized with a pro-peptide, a catalytic domain, a hinge or a linker region, and a hemopexin-like C-terminal domain. Native form of MMP7 is activated by a variety of extracellular activation mechanisms including autolytic activation but these mechanisms are not well defined. As these mechanisms are not well known, recombinant expression in T/NK cells is not guaranteed to enable processing to the mature form, which facilitates the ECM degradation. Furthermore, MMPs are expressed at relatively low levels in normal conditions and are usually upregulated when degradation is required. To circumvent this challenge, the mature form of MMP8 was expressed that does not need the removal of the pro-peptide. MMP8 promotes a complete remodeling of the tumor microenvironment through multiple mechanisms including promoting immune cell infiltration, remodeling metabolism and oxygenation, and tumor cell signaling. Without the support of the ECM architecture, solid tumors will be compromised through multiple mechanisms making them sensitive to CAR T cell mediated regression.

Embodiments include a genetically modified immune cell with a mammalian cell expression vector containing a nucleic acid encoding a mature form of a matrix metallopeptidase. The matrix metallopeptidase can be a matrix metallopeptidase 1, matrix metallopeptidase 8, matrix metallopeptidase 13, matrix metallopeptidase 18, or combinations thereof. In certain embodiments, the matrix metallopeptidase is a mature matrix metallopeptidase 8. The immune cell can be modified by introduction of a nucleic acid encoding the matrix metallopeptidase. The immune cell can be modified by transfection of a mRNA encoding the matrix metallopeptidase. This method reduces the likelihood that the gene encoding the matrix metallopeptidase will integrate into the genome of the cell. In some embodiments, the mRNA is capped using 7-methyl-guanosine. In some embodiments, the mRNA may be polyadenylated. The immune cell can be a lymphocyte, which can be a T cell, a NK cell, a NK T cell, or B cell. The immune cell can be a myeloid cell. The immune cell can be isolated from peripheral blood or from human tumors.

Embodiments include a genetically modified immune cell with a mammalian cell expression vector containing a nucleic acid encoding a mature form of a matrix metallopeptidase and a second mammalian cell expression vector containing a second nucleic acid encoding a chimeric antigen receptor. In particular embodiments, an expression construct encoding a matrix metallopeptidase can be a part of polycistronic construct encoding the matrix metallopeptidase and the chimeric antigen receptor that are simultaneously expressed in the cell. Embodiments include a genetically modified immune cell with a mammalian cell expression vector containing a nucleic acid encoding a mature form of a matrix metallopeptidase and a second mammalian cell expression vector containing a second nucleic acid encoding a cytokine. In particular embodiments, an expression construct encoding a matrix metallopeptidase can be a part of polycistronic construct encoding the matrix metallopeptidase and the cytokine that are operably linked for expression in the cell.

The mammalian cell expression vector containing a nucleic acid encoding a mature form of a matrix metallopeptidase can also include one or more promoters driving expression of the matrix metallopeptidase. In certain embodiments, the promoter is designed to support constitutive expression of the matrix metallopeptidase in the immune cells. For example, gene expression from retroviral vectors can be driven by either the retroviral long terminal repeat (LTR) promoter or by cellular or viral promoters located internally in an LTR-deleted self-inactivating vector design. These promoter can also include constitutive promoters such as the cytomegalovirus early (CMV) promoter or the SV40 early promoter as well as inducible promoters such as the tetracycline-inducible promoter. Examples of enhancers and promoters that can be used to drive expression of a nucleic acid sequence encoding one or more matrix metallopeptidase or the chimeric antigen receptor provided herein include, without limitation, a CMV enhancer sequence, a CMV promoter sequence, a CAG enhancer sequence, a CAG promoter sequence, a RSV enhancer sequence, a RSV promoter sequence, a hPGK promoter, a RPBSA promoter, a Ef1 alpha enhancer sequence, a Ef1 alpha promoter sequence, a ubiquitin enhancer sequence, a ubiquitin promoter sequence, adenovirus enhancer sequences, adenovirus promoter sequences, tetracycline inducible promoters, and salicylic acid inducible promoter.

Composition containing these genetically modified immune cells are formulated to be suitable for administration to a subject. Generally, the composition is free of contaminants that are capable of eliciting an undesirable response within the subject. These compositions can be designed for administration to subjects in need thereof via a number of different routes of administration including intravenous, intratumoral, buccal, intraperitoneal, intradermal, intramuscular, subcutaneous, and the like. The immune cells can be T-cells, tumor infiltrating lymphocytes, macrophages, or NK cells, each of the foregoing with and without being modified to express a chimeric antigen receptor.

Embodiments include methods of treating a solid tumor in a patient by administering a composition containing chimeric antigen receptor-expressing T-cells modified to express a matrix metallopeptidase. One such method includes administering a composition containing chimeric antigen receptor-expressing T-cells modified to express a mature matrix metallopeptidase 8. The solid tumor can be associated with brain cancer, breast cancer, lung cancer, colorectal cancer, prostate cancer, or cervical cancer. In particular embodiments, the patient has received, is receiving, or will receive an additional cancer treatment, such as one that includes chemotherapy, immunotherapy, radiation, surgery, hormone therapy, or a combination thereof. In certain embodiments, the method further includes the step of administering a chemotherapeutic agent. The chemotherapeutic agent can be one or more of doxycycline, doxorubicin, gefitinib, erlotinib, everolimus, afatinib, and crizotinib. In certain embodiments, the method further includes a step of administering an immunotherapeutic agent. The immunotherapeutic agent can be an anti-PD1 antibody, anti-PDL1 antibody, anti-CTLA4 antibody, anti-LAG3 antibody, or an anti-TIM-3 antibody or combinations thereof.

Embodiments include methods of treating a solid tumor in a patient by administering a composition containing chimeric antigen receptor-expressing NK cells modified to express mature matrix metallopeptidase. One such method includes administering a composition containing chimeric antigen receptor-expressing NK-cells modified to express a mature matrix metallopeptidase 8. The solid tumor can be associated with brain cancer, breast cancer, lung cancer, colorectal cancer, prostate cancer, or cervical cancer. In particular embodiments, the patient has received, is receiving, or will receive an additional cancer treatment, such as one that includes chemotherapy, immunotherapy, radiation, surgery, hormone therapy, or a combination thereof. In certain embodiments, the method further includes a step of administering a chemotherapeutic agent. The chemotherapeutic agent can be one or more of doxycycline, doxorubicin, gefitinib, erlotinib, everolimus, afatinib, and crizotinib. In certain embodiments, the method further includes a step of administering an immunotherapeutic agent. The immunotherapeutic agent is an anti-PD1 antibody, anti-PDL1 antibody, anti-CTLA4 antibody, anti-LAG3 antibody or an anti-TIM-3 antibody or combinations thereof.

As used herein, the terms “treatment,” “treating,” and “treat” refer to any indicia of success in the treatment or amelioration of cancer or a pre-cancerous condition, including any objective or subjective parameter such as abatement, remission, diminishing of symptoms or making the cancer or a pre-cancerous condition more tolerable to the subject, slowing in the rate of degeneration or decline, making the final point of degeneration less debilitating, and/or improving a subject’s physical or mental well-being. “Treatment” does not necessarily indicate complete eradication or cure of the disease or condition, or associated symptoms thereof. The terms “administer,” “administering,” and “administration” refer to introducing a compound, a composition, or an agent (e.g., genetically modified immune cell with a mammalian cell expression vector containing a nucleic acid encoding a mature form of a matrix metallopeptidase or a chemotherapeutic agent) into a subject or subject, such as a human. As used herein, the terms encompass both direct administration, e.g., self-administration or administration to a subject by a medical professional, and indirect administration, such as the act of prescribing a compound, composition, or agent. As used herein, the term “matrix metallopeptidase” includes any suitable enzymatically active portion, a homolog, or a variant thereof, such as a codon optimized sequence or any sequence corresponding to SEQ ID NO. 1 or SEQ ID NO. 2 and containing one or more different structural or chemical modifications, before and/or after codon optimization.

Most CAR T-cell therapies involve ex vivo expansion before their infusion in cancer patients. This persistent ex vivo expansion of CAR T-cells could alter their effector functionality and reduce their efficacy in decimating solid tumors. The effector functions of homing of immune-effector cells to the tumor and their ability to penetrate the ECM surrounding the solid tumors, if compromised by their persistent ex vivo expansion, could severely impact the prognosis of cancer patients. Embodiments include a composition containing a population of immune cells modified to alter expression of a matrix metallopeptidase. The MMP expressed can be mMMP8. Embodiments include methods of treating a solid tumor in a patient. One such method includes administering a composition comprising chimeric antigen receptor-expressing T-cells modified to express matrix metallopeptidase 8. Another such method includes administering a composition comprising chimeric antigen receptor-expressing NK cells modified to express matrix metallopeptidase 8. In certain embodiments, the method further includes the step of administering a chemotherapeutic agent. In certain embodiments, the chemotherapeutic agent is doxycycline.

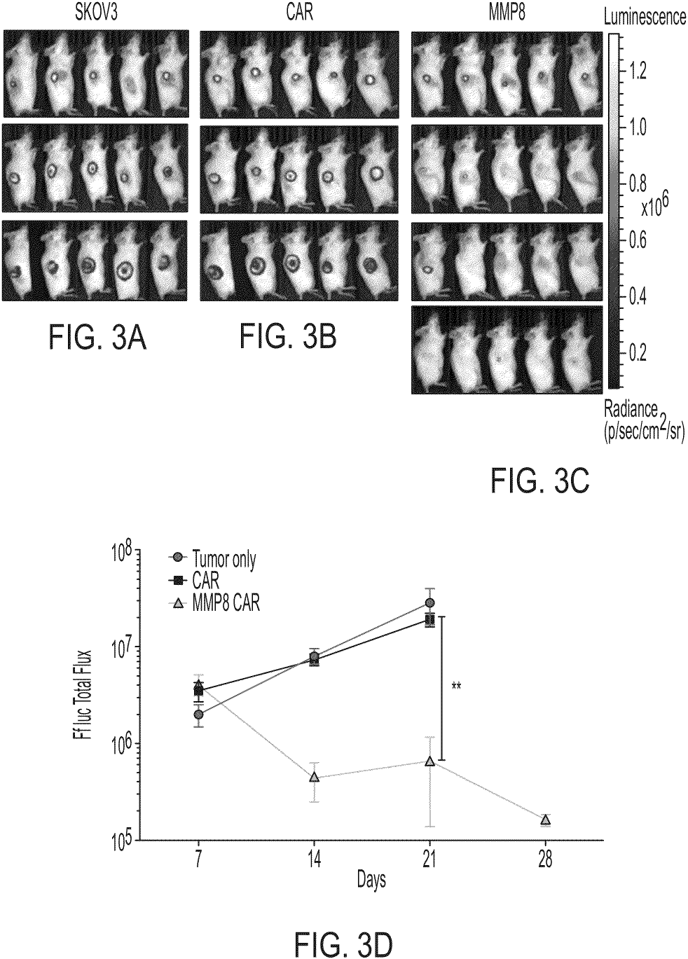

The ECM degradative capability of ex vivo expanded T-cells was examined. Data herein indicated that persistent in vitro culture of activated human T-cells resulted in downregulation of MMP8 both at transcript and protein level. Using Matrigel based migration assay, it was observed that downregulation of MMP8 in T-cells concomitantly compromised their ability to degrade and migrate across Matrigel. Given the role of MMP8 in the digestion of ECM, loss of MMP8 in CAR T-cells compromised their ability to penetrate the ECM and promoted tumor regression. To address this problem, CAR T-cells were genetically manipulated to express MMP8. These modified cells were evaluated for their ability to rescue the effects of endogenous MMP8 downregulation. For example, NALM6 and SKOV-3 tumor cells were used as the tumor model. SKOV-3 cells genetically modified to express CD19 antigen have been previously described. The modified MMP8 overexpressing CAR T-cells can efficiently kill NALM6 and SKOV-3 tumor cells embedded in Matrigel compared to CAR T-cells alone. As compared to either target cells or CAR T-cells embedded in Matrigel, MMP8 overexpressing CAR T-cells were more proficient than CAR T-cells alone in migrating across the Matrigel and killing their targets. These results were confirmed in the in vivo studies. In a tumor model using SKOV-3, MMP8 overexpressing CAR T-cells promoted complete tumor remission in NSG™ mice. These mice carry two mutations on the NOD/ShiLtJ genetic background-severe combined immune deficiency (scid) and a complete null allele of the IL2 receptor common gamma chain (IL2rg^{null}). The scid mutation is in the DNA repair complex protein Prkdc and renders the mice B and T cell deficient. The IL2rg^{null} mutation prevents cytokine signaling through multiple receptors, leading to a deficiency in functional NK cells. All the CAR T-cell alone treated mice developed tumors similar to the control group. The genetically modified CAR T-cells expressing MMP8 provide novel treatment options against solid tumors by helping to overcome the existing limitations of CAR T-cell therapy against solid tumors in part attributed to the ECM. MMP8 deficiency in in vitro expanded CAR T-cells may limit their antitumor activity in stroma-rich solid tumors. The mMMP8-CAR T cells demonstrated superior migration and killing ability of multiple CD19⁺ tumor models compared to parental CAR T cells. For example, the in vivo efficacy of these genetically modified CAR T cells was tested in NSG™ mice engrafted with SKOV3-CD19 tumors. Unlike the parental CAR T cells that only enabled transient delay in tumor growth leading to the death of the mice, mice administered MMP8-CAR T cells rejected the tumors and survived tumor free for >100 days. The untreated mice with the tumor and CD19R-41BBZ CAR T cell treated mice developed tumors and died by day 25 or were euthanized due to high tumor burden (FIGS. 3A - 3C).

Embodiments of the present disclosure include genetically modified CAR T-cells expressing mMMP8. These cells have an improved capacity to degrade ECM and increase their ability to infiltrate deep inside the tumor mass and promote the remission of the tumor. This therapeutic regimen potentiates the antitumor activity of CAR T-cell therapy in patients with stroma-rich solid tumors. Embodiments include methods of preparing a CAR T cell product that decreases or overcomes primary resistance to CAR T cell treatment comprising improving the CAR T cells by co-expressing a mMMP8 under constitutive or inducible promoters in the CAR T cells.

### EXAMPLES

The following examples are provided to illustrate, but not to limit, the scope of the claimed embodiments.

### Example 1 - Long Term Expansion of T-Cells Reduced Their Invasion of ECM and Loss of the Enzyme MMP8

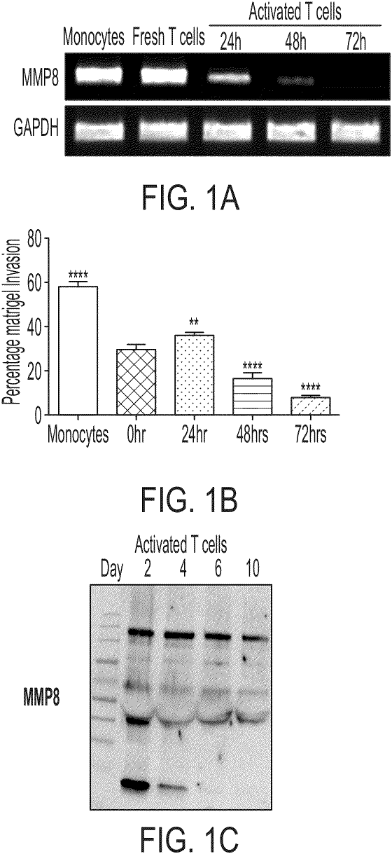

Cancer immunotherapy for solid tumors hinges on the ability of immune cells to infiltrate to the tumor site and subsequently degrade the ECM to gain access to the tumor cells and exert antitumor effects. For example, CAR T-cell therapy involves ex vivo expansion of antigen-reactive T-cells over a couple of weeks. This exponential in vitro expansion may compromise their effector functions and render them unfit for tumor regression. The ECM degradation capability of ex vivo expanded human T-cells was examined. The RNA transcript levels of MMP8 in resting T-cells were compared with that of T-cells activated for different intervals of time by RT-PCR. FIG. 1A is a photographic representation of the data from a RT PCR assay of monocytes and T-cells upon activation at different time intervals. Even short-term ex vivo expansion of activated T-cells resulted in a marked decrease in MMP8 transcript level (FIG. 1A).

The functional significance of attenuated MMP8 transcript in activated T-cells was investigated. A trans-well migration assay across Matrigel was performed. The invasive capability of freshly isolated T-cells was compared with that of T-cells activated for different intervals of time. The migratory ability of T-cells was directly related to their MMP8 transcript levels. FIG. 1B is a graphical representation of the results from a Matrigel invasion assay for monocytes and activated T-cells. Consequently, resting T-cells and briefly activated T-cells for 24 hours, showed superior invasion of Matrigel compared to T-cells activated beyond 48 hours.

The MMP8 protein levels secreted by activated T-cells were evaluated over different intervals of time. FIG. 1C is a photographic representation of the data from a Western Blot analysis for secreted MMP8 expression in conditioned media of activated T-cells. Western blotting of conditioned media indicated that MMP8 protein levels in the media significantly decreased when the T-cells were activated over 48 hours. The ex vivo expansion of T-cells results in downregulation of MMP8 both at transcript and protein level and is associated with their reduced migration across Matrigel.

### Example 2 - CAR T-Cells Co-Expressing mMMP8 Have Enhanced Capacity to Degrade ECM

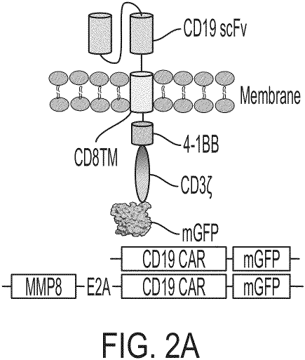

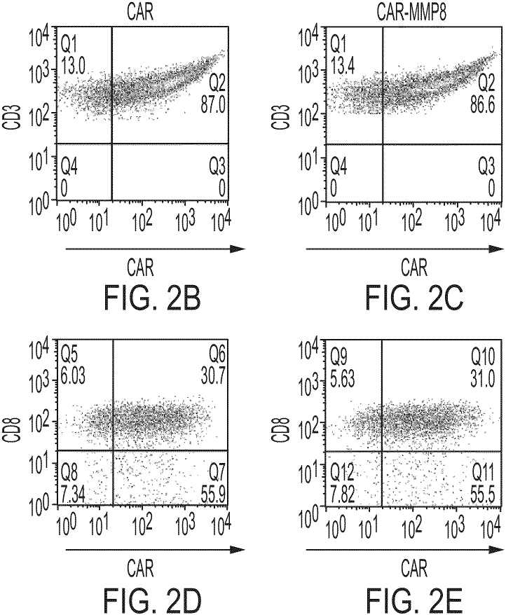

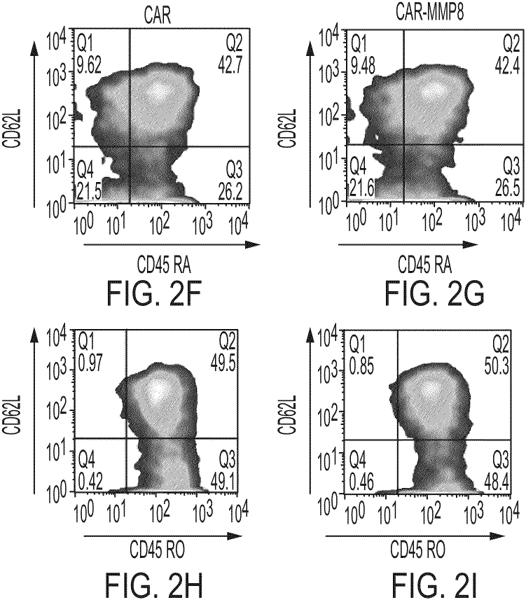

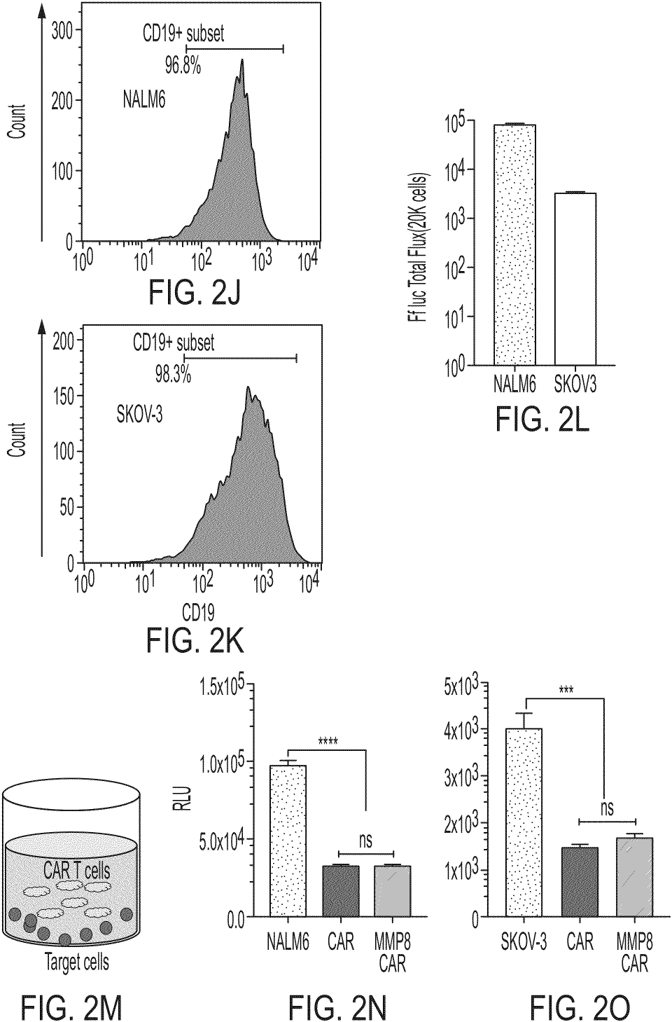

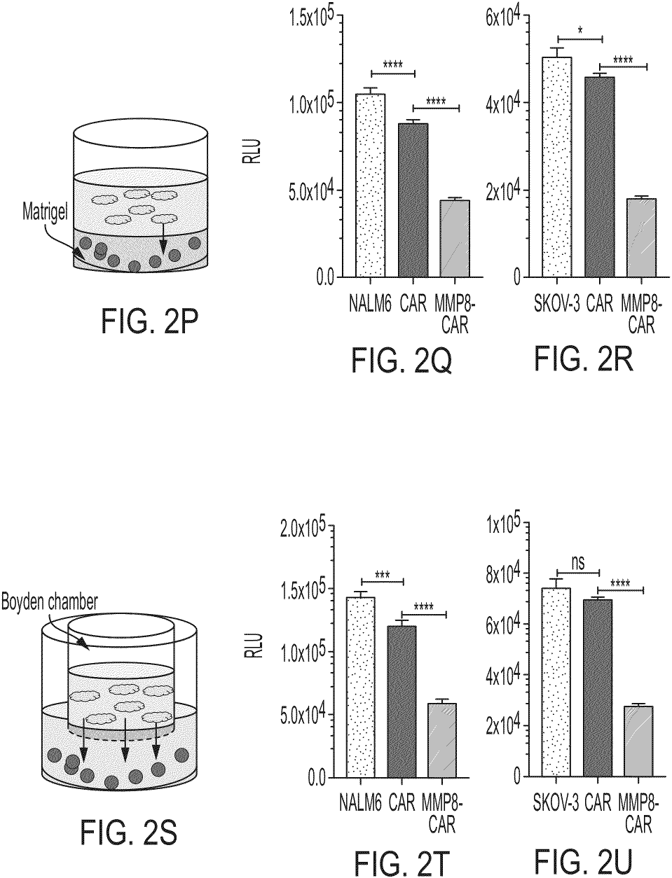

CAR T-cells engineered to express MMP8 were designed to improve the invasive capacity of in vivo expanded CAR T-cells. To evaluate the functional significance of MMP8 in CAR T-cells, CAR constructs were generated. CD19 CAR single-chain variable fragment (scFv) was fused to the CD8TM domain followed by 4-1BB and CD3ζ. To monitor the transduction efficiency of the CAR in T-cells, monomeric GFP (mGFP) was fused to the CD3z end of the CAR. FIG. 2A is a diagrammatic representation of the CAR construct containing the MMP8-CD19R-4-1BB-CD3z-mGFP. The CAR construct was cloned into a retroviral plasmid as CD19 CAR_mGFP and Human mMMP8 CAR as hmMMP8-E2A-CD19 CAR_mGFP. Subsequently, activated T-cells were transduced with the viral particles to generate CAR T-cells. CAR T-cells were expanded for 10 days in presence of cytokines IL7 and IL15.

FIGS. 2B - 2E are representations of the phenotyping of CAR T-cells vs mMMP8-CAR T-cells. FIG. 2B and FIG. 2C are flow plots capturing the expression of the CD3 in CAR T-cells and mMMP8-CAR T-cells, respectively. FIG. 2D and FIG. 2E are flow plots capturing the expression of the CD8 in CAR T-cells and mMMP8-CAR T-cells, respectively. Fluorescent intensity are the units for all flow plots. FIG. 2F and FIG. 2G are representations of flow cytometry evaluation of CD62L and CD45 RA in CAR T-cells and mMMP8-CAR T-cells, respectively, and FIG. 2H and FIG. 2I are representations of flow cytometry evaluation of CD62L and CD45 RO in CAR T-cells and mMMP8-CAR T-cells, respectively. On day 10, phenotyping of those CAR T-cells revealed that MMP8 overexpression did not alter the memory phenotype of CAR T-cells compared to CAR T-cells alone as shown by flow cytometry evaluation of CD62L, CD45RO, and CD45RA memory markers. NALM6 (B cell precursor leukemia cell line) and SKOV-3 (ovarian cancer cell line) were used as the tumor cell models. SKOV-3 cells expressing CD19 antigen have been previously described. Both these cell lines were genetically modified to express Firefly luciferase (FF-Luc) to facilitate quantification in in vitro and in vivo experiments. Surface staining for CD19 on NALM6 and SKOV-3 was evaluated with flow cytometry and FF-Luc expression was evaluated using the D-luciferin substrate as luminescence using TopCount plate reader. FIG. 2J and FIG. 2K are graphical representations of the results from the phenotyping of NALM6 and SKOV-3 cells for surface CD19 expression. FIG. 2L is a graphical representation of FF-Luc expression in NALM6 and SKOV-3 cells.

To evaluate the in vitro efficacy of the different CAR constructs to kill targets, coculture studies of targets and effectors were performed. FF-Luc Nalm6 cell or FF-Luc SKOV-3 were cocultured with either CAR T-cells or MMP8 expressing CAR T-cells in 3:1 E:T ratio in 96 well. After 4 hours, D-luciferin substrate was added to each well and the luminescence was measured with TopCount plate reader. FIG. 2M is a diagrammatic representation of the set-up for the killing assay upon co-culture of effector and targets in 96-well plate using D-luciferin substrate. FIG. 2N and FIG. 2O are graphical representations of the results from the killing assay upon co-culture of CAR T-cells and MMP8-CD19R-41BBz CAR T cells in the presence of NALM6 and SKOV-3 cells, respectively. There was no significant difference in the killing ability of CAR T-cells vs MMP8-CAR T-cells. As expected, MMP8 does not appear to offer an advantage in settings where targets and effectors are not separated by extracellular matrix.

To perform a functional assay using MMP8, Matrigel, a known substrate of MMP8, was used. Next, 0.25×10⁵ NALM6 cells were seeded in 25µl Matrigel in 96-well plate. The Matrigel was allowed to solidify at 37° C. for 30 minutes. Next, 100 µl of CAR T-cells were laid at a concentration of 0.75×10⁶ cells/ml on top of the Matrigel enclosed target cells. FIG. 2P is a diagrammatic representation of the set-up for the killing assay in Matrigel performed in 96-well plate. The plate was incubated for 18 hours. D-luciferin substrate was subsequently added to quantify the live target cells. FIG. 2Q and FIG. 2R are graphical representations of the results from the killing assay of CAR T-cells and MMP8-CD19R-41BBz CAR T cells laid on top of the solidified Matrigel containing NALM6 and SKOV-3 cells, respectively. MMP8 expressing CAR T-cells were more efficient in killing the NALM6 tumor cells in Matrigel compared to CAR T-cells without MMP8. Similarly, MMP8 CAR T-cells were able to kill SKOV-3 cells more efficiently than CAR T-cells alone. FIG. 2E is a graphical representation of the results from the killing assay in Matrigel performed in 96-well plate.

Next, the ability of CAR T-cells to pass through Matrigel in the Boyden chamber and kill the targets was tested. Tumor cells were seeded in Matrigel and CAR T-cells were laid on top of the solidified Matrigel. FIG. 2S is a diagrammatic representation of the set-up for the killing assay using Boyden chamber. Tumor cells were seeded in the bottom chamber and CAR T-cells were plated in the Matrigel coated upper chamber. FIG. 2T and FIG. 2U are graphical representations of the results from the killing assay of CAR T-cells and MMP8-CD19R-41BBz CAR T cells in the in the Matrigel coated upper chamber above NALM6 and SKOV-3 cells, respectively, seeded in the bottom chamber. MMP8 expressing CAR T-cells exhibited greater migratory capability across Matrigel compared to CAR T-cells alone. In conclusion, MMP8 improved the migration of in vitro expanded CAR T-cells compared to CAR T-cells alone.

CD19 CAR T-cells co-expressing MMP8 show enhanced tumor infiltration and improve overall survival in xenograft tumor models. Xenografts of SKOV-3 tumor cell line were established in NSG™ mice in presence of Matrigel to enable the formation of structured solid tumors. Mice were subcutaneously injected with 0.5×10⁶ into the right flank. One week later mice received 5×10⁶ CAR T-cells or MMP8-CAR T-cells intravenously. Every week, anesthetized mice were injected with D-Luciferin and underwent bioluminescent imaging in a lateral position using a Xenogen IVIS 100 series system. FIGS. 3A - 3C are photographs representation of the in vivo imaging data demonstrating the enhancement of tumor infiltration and improvement of overall survival in xenograft tumor models, both in control mice (FIG. 3A) and in mice with CD19 CAR T-cells (FIG. 3B) and with CD19 CAR T-cells co-expressing mMMP8 (FIG. 3C) over the experimental time period. FIG. 3D is a graphical representation of the tumor growth curve in control mice and in mice with CD19 CAR T-cells and with CD19 CAR T-cells co-expressing mMP8. As shown in FIGS. 3A - 3D, mice injected with MMP8-CAR showed complete regression of the tumor in all mice and survived beyond 60 days. On the other hand, CAR T-cell alone treated mice did not show any significant difference in tumor burden compared to no treatment mice, all of which died within a month of tumor cell injection.

### Example 3 - NK T-Cells Co-Expressing mMMP8 Have Enhanced Capacity to Degrade ECM

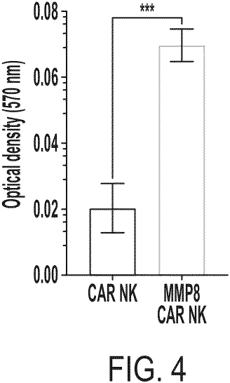

The construct for expression in NK cells was the same as used in Example 2. NK cells were transduced with CAR or MMP8 CAR and tested for their efficacy to migrate across Matrigel in a transwell based assay. CAR NK and MMP8-CAR NK cells were seeded in Matrigel coated upper compartment of Boyden chamber. After 18 hours, the top chamber was removed and the number of cells that migrated to lower chamber were quantified using a MMT reagent. As shown in FIG. 4, MMP improved the migratory capabilities of NK cells across Matrigel. FIG. 4 is a graphical representation of the results from the killing assay using Boyden chamber. MMP8 expressing CAR NK-cells exhibited greater migratory capability across Matrigel compared to CAR NK-cells alone.

Taken together the in vitro and in vivo data suggest that long term in vitro expanded immune cells show reduced migration across ECM in part attributed to MMP8 downregulation and that overexpression of MMP8 in immune cells improved their migration ability and to promote tumor regression. The downregulation of MMP8 in CAR immune cells can limit their antitumor activity in solid tumors. Next-generation immune cells genetically modified to secrete MMP8 can enable efficient immune-cell trafficking to stroma-rich solid tumors leading to enhanced tumor control and survival.

While various specific embodiments/aspects have been illustrated and described, it will be appreciated that various changes can be made without departing from the spirit and scope of the disclosure.

What is claimed is:

1. A composition comprising genetically modified immune cells expressing a mature form of a matrix metallopeptidase.

2. The composition of claim 1, wherein the matrix metallopeptidase is a matrix metallopeptidase 8.

3. The composition of claim 1, wherein the genetically modified immune cells contain a mammalian cell expression construct with a nucleic acid encoding the matrix metallopeptidase.

4. The composition of claim 1, wherein the genetically modified immune cells contain a mRNA construct for expression of the matrix metallopeptidase.

5. The composition of claim 1, wherein the genetically modified immune cells are one or more of T cells, Natural Killer cells, Natural Killer T cells, or B cells.

6. The composition of claim 1, wherein the genetically modified immune cells are myeloid cells.

7. The composition of claim 1, wherein the genetically modified immune cells are further modified to express a chimeric antigen receptor.

8. The composition of claim 1, wherein the immune cells are isolated from peripheral blood or from human tumors.

9. The composition of claim 1, wherein the genetically modified immune cells are further modified to express a cytokine.

10. A method of treating a solid tumor in a patient, the method comprising:
administering a composition comprising chimeric antigen receptor-expressing T-cells modified to express a mature form of a matrix metallopeptidase 8.

11. The method of claim 10, wherein the solid tumor is associated with brain cancer, breast cancer, lung cancer, colorectal cancer, prostate cancer, or cervical cancer.

12. The method of claim 10, further comprising a step of administering a chemotherapeutic agent.

13. The method of claim 12, wherein the chemotherapeutic agent is one or more of doxycycline, doxorubicin, gefitinib, erlotinib, everolimus, afatinib, and crizotinib.

14. The method of claim 10, further comprising a step of administering an immunotherapeutic agent.

15. The method of claim 14, wherein the immunotherapeutic agent is one or more of an anti-PD1 antibody, anti-PDL1 antibody, anti-CTLA4 antibody, anti-LAG3 antibody, and an anti-TIM-3 antibody.

16. A method of treating a solid tumor in a patient, the method comprising:
administering a composition comprising chimeric antigen receptor-expressing natural killer cells modified to express a mature form of a matrix metallopeptidase 8.

17. The method of claim 16, further comprising a step of administering a chemotherapeutic agent.

18. The method of claim 17, wherein the chemotherapeutic agent is one or more of doxycycline, doxorubicin, gefitinib, erlotinib, everolimus, afatinib, and crizotinib.

19. The method of claim 16, further comprising a step of administering an immunotherapeutic agent.

20. The method of claim 19, wherein the immunotherapeutic agent is one or more of an anti-PD1 antibody, anti-PDL1 antibody, anti-CTLA4 antibody, anti-LAG3 antibody, and an anti-TIM-3 antibody.
